(19) United States
(12) Patent Application Publication
(10) Pub. No.: US 2012/0022392 A1
(43) Pub. Date: Jan. 26, 2012

(54) Correlating Frequency Signatures To Cognitive Processes

(75) Inventors: Eric C. Leuthardt, St. Louis, MO (US); Charles Gaona, Swansea, IL (US); Mohit Sharma, St. Peters, MO (US)

(73) Assignee: WASHINGTON UNIVERSITY IN ST. LOUIS, St. Louis, MO (US)

(21) Appl. No.: 13/189,021

(22) Filed: Jul. 22, 2011

Related U.S. Application Data
(60) Provisional application No. 61/366,728, filed on Jul. 22, 2010.

(51) Int. Cl. A61B 5/0476

(52) U.S. Cl. 600/544

(57) ABSTRACT

Determining an intended action based on one more brain signal frequencies includes establishing communication with one or more electrodes for sensing brain signals of a subject, and acquiring the brain signals via the electrodes while the subject performs at least one cognitive task, wherein the acquired brain signals having a plurality of frequencies associated therewith. A physiologic change at one or more of the plurality of frequencies may then be identified from the acquired brain signals, and the one or more of the plurality of frequencies are associated with the cognitive task.

### CROSS REFERENCE TO RELATED APPLICATIONS

This application claims the benefit of Provisional Patent Application Ser. No. 61/366,728, entitled “CORRELATING FREQUENCY SIGNATURES TO COGNITIVE PROCESSES”, which was filed on Jul. 22, 2010 and which is hereby incorporated by reference in its entirety.

### BACKGROUND

Embodiments described herein relate generally to a brain computer interface and, more particularly, to detecting non-uniform changes in gamma frequencies that occur within the brain and that depend on an intended cognitive action.

Clinical use of ECoG gamma band power changes in electrophysiological environments has shown at least two known issues. First, power changes in frequency ranges below 250 Hertz (Hz) have not been evaluated. Second, at least some known ECoG systems assume that such ECoG gamma band power changes are uniform. Moreover, at least some known ECoG systems evaluate all frequencies above a lower threshold as a single response. Other ECoG systems examine power changes in a specific range of frequencies, such as between 70 Hz and 100 Hz. Still other ECoG systems correlate behavior with uniform and broadband (e.g., 5-200 Hz) increases in power putatively caused by increases in asynchronous neuronal activity.

### BRIEF DESCRIPTION OF THE DRAWINGS

The embodiments described herein may be better understood by referring to the following description in conjunction with the accompanying drawings.

FIG. 1 is a block diagram of an exemplary brain computer interface (BCI).

FIG. 2 is a block diagram of signal acquisition circuitry that may be used with the BCI shown in FIG. 1.

FIG. 3 is a block diagram of signal analysis circuitry that may be used with the BCI shown in FIG. 1.

FIG. 4 is a flowchart that illustrates an exemplary method for controlling a device based on one more brain signal frequencies using the BCI shown in FIG. 1.

FIGS. 5A-5D are graphs illustrating test results of seven right-handed subjects that clinically required the placement of electrode arrays over the surface of their left frontal and/or temporal cortex.

FIG. 6 is a graph illustrating a percentage of the seven subjects that exhibited significant power change by frequency.

FIGS. 7A-7C illustrate an exemplary experimental setup for use with the BCI shown in FIG. 1.

FIGS. 8A-8D are graphs illustrating a means of quantifying a non-uniform and narrowband nature of the evoked spectra.

FIGS. 9A-9F are graphs showing individual subject normalized spectral responses that illustrate activation flips for a subset of the seven subjects.

FIGS. 10A-10F are graphs showing normalized spectra for a single channel across all six cognitive tasks for the same subject shown in FIG. 8C.

FIGS. 11A-11F are graphs showing normalized spectra computed using Fast Fourier Transforms (FFT) instead of the autoregressive method used to generate the spectra of FIGS. 10A-10F.

FIG. 12 is a set of graphs showing activation flips for the seven subjects.

FIG. 13 illustrates consolidated cortical activation plots for the seven subjects.

FIG. 14 illustrates cortical activation plots for a single subject.

FIG. 15 is a table illustrating quantitative measures of trends observed from FIGS. 12-14.

FIG. 16 illustrates a set of normalized spectra that were defined while a subject performed a center joystick task.

### DETAILED DESCRIPTION

Embodiments of the invention enable detection of distinct narrowband, task-evoked power changes in multiple independent frequency bands for use in determining an intended cognitive task. In some embodiments, the power changes are detected in frequency bands ranging from 0.1 Hz to 550 Hz, or above 550 Hz in other embodiments. In some embodiments, the power changes are detected in frequency bands ranging from 30 Hz to 550 Hz. Moreover, some embodiments of the disclosure enable detection of task-evoked phase changes and/or task-evoked event-related potentials.

In some embodiments, an implantable brain-computer interface (BCI) controls, for example, a prosthetic hand for a subject with a motor control impairment such as a stroke by analyzing frequency signatures of cortical signals acquired from the unaffected portions of the brain. In some embodiments, this is achieved by detecting changes to the frequency signatures that are associated with intended actions by the subject. The changes are translated to support independent thought-driven device control. The cortical signals may be acquired, for example, from one or more of the primary motor cortex, the premotor cortex, the frontal lobe, the parietal lobe, the temporal lobe, and the occipital lobe of the brain.

To facilitate understanding of the embodiments described herein, certain terms are defined below.

In some embodiments, the term “electrocorticography” and the acronym “ECoG” refer generally to a technique that involves recording surface cortical potentials from either epidural or subdural electrodes.

In some embodiments, the term “brain computer interface” and the acronym “BCI” refer generally to signal-processing circuitry that acquires input in the form of raw brain signals and converts the brain signals to a processed signal that is output to a device for storage and/or further analysis. Moreover, in some embodiments, the term “BCI system” refers generally to a number of components, including a BCI, that translates raw brain signals into control of a device.

In some embodiments, the term “device” refers generally to equipment or a mechanism that is designed to provide a special purpose or function. Exemplary devices including, but are not limited to, a cursor on a video monitor, computer software, environmental controls, entertainment devices, prosthetics, beds, and mobility devices such as wheelchairs or scooters. Moreover, the term also includes input devices that are used to control other devices such as those that are listed above. Exemplary input devices include, but are not limited to, wheels, joysticks, levers, buttons, keyboard keys, trackpads, and trackballs.

Embodiments described herein acquire and analyze signals for physiologically relevant information at frequencies as high as 550 Hz, or higher. Synchronously acquiring neuronal activity enables the evoked spectra to demonstrate narrowband changes that occur in distinct frequency bands.

The cortical signals may be obtained from one or more of ECoG signals, electroencephalography (EEG) signals, local field potentials, single neuron signals, magnetoencephalography (MEG) signals, mu rhythm signals, beta rhythm signals, low gamma rhythm signals, high gamma rhythm signals, and the like. Moreover, the ECoG signals, EEG signals, local field potentials, and/or MEG signals may include one or more of mu rhythm signals, beta rhythm signals, low gamma rhythm signals, and high gamma rhythm signals. The signal data is converted into the frequency domain and spectral changes are identified with regards to frequency, amplitude, phase, location, and timing. The embodiments described herein enables high signal resolution associated with ECoG, for example, to reveal aspects of cortical signal processing that is unavailable with noninvasive means.

Known ECoG studies have not identified distinct narrowband, high frequency evoked power change patterns in their findings. For example, differences in behavioral tasks, data collection methods, and analysis techniques may have obscured such patterns. In addition, many ECoG studies have utilized experimental paradigms that are designed to illuminate cortical changes that are caused by subtle differences in cognitive behaviors, such as phonological processing, semantic processing, lexical processing, and the like. Such paradigms often purposely focus on cortical responses to input stimuli with relatively simple responses, such as a button press, or with passive stimulation alone. While the differences in high frequency activation may have been present, they may have been too subtle to notice and/or within the current uniform view of gamma power changes, and may therefore been considered irrelevant. Additionally, studies of relatively simple motor tasks, such as hand clasping or finger movements, that have reported wideband power increases that are correlated to motor behavior may involve different physiologies. Functional imaging studies of finger movements implicate much smaller regions of BOLD signal change than those of the language tasks described herein. A difference between a more focal versus a more networked cortical process may result in different electrophysiological responses. Thus, broadband responses to motor tasks may also be task specific and location specific, but may not generalize to other tasks or cortical areas.

Signal to noise ratios and frequency analysis techniques may also explain why other research has not reported on the high frequency behavior described herein. For example, the raw power spectral density of electrical cortical activity decreases exponentially in proportion to the observation frequency such that, when analyzing high frequencies, practices that enhance the signal to noise ratio are desirable. ECoG recordings described herein used intracranial and non-cortical (skull facing) reference electrodes that are less susceptible to noise than scalp or cortical electrodes used for other recording techniques. Moreover, analyzing power changes in preselected frequency ranges, such as between 80 Hz and 100 Hz, generally does not reveal band-specific power changes either within or outside of those boundaries without further analysis. Linear time-frequency analysis techniques, such as wavelet and Fourier transforms, are commonly used, but inherently trade off time resolution and frequency resolution. Selecting analysis parameters that favor a finer time resolution may obscure narrowband changes because of coarse frequency resolutions at higher ranges.

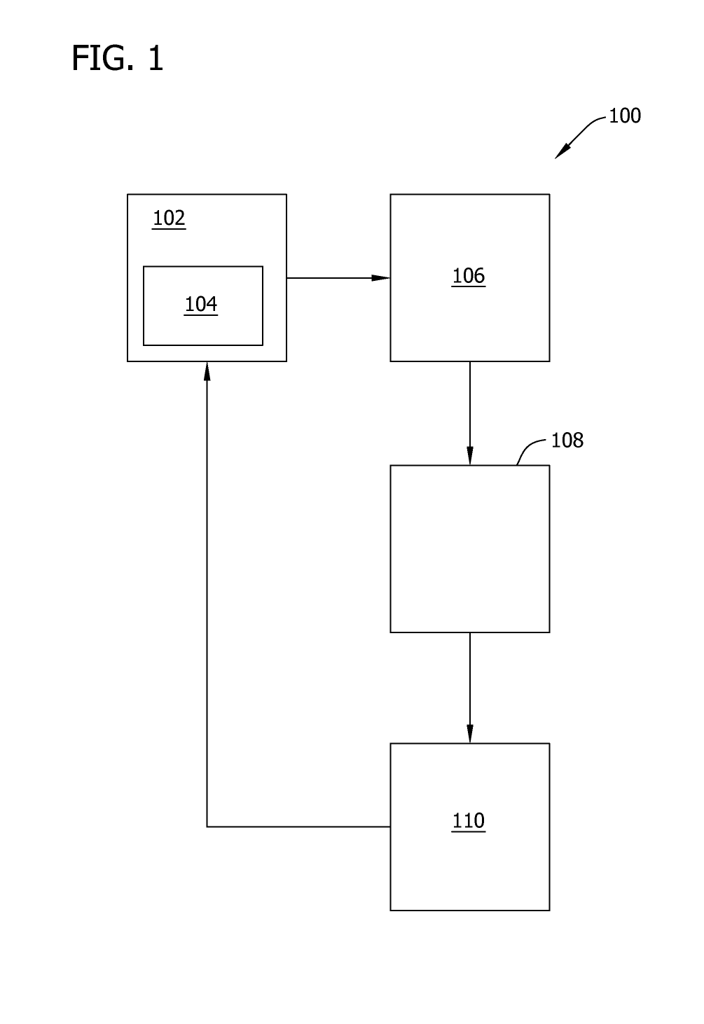

FIG. 1 is a block diagram of an exemplary brain computer interface (BCI) 100 for use acquiring brain signals from a subject's brain 102, translating the brain signals into a control signal, and performing an intended action associated with the brain signals. In some embodiments, BCI 100 includes an implantable electrode array 104 that may be positioned either under the dura mater (subdural) or over the dura mater (epidural). In the example of FIG. 1, electrode array 104 is subdural. Electrode array 104 includes a plurality of electrodes (not shown in FIG. 1), such as ECoG electrodes that acquire brain signals from a surface of the brain and generate a raw ECoG signal. Electrode array 104 may be arranged in an 8×8 or 6×8 grid, although other grid arrangements are contemplated. The individual electrodes have a diameter of approximately 4 millimeters (mm) and are composed of, for example, platinum iridium discs. The electrodes are spaced approximately 1 centimeter apart and are encapsulated in silastic sheets, such that separate four-electrode strips were created and implanted facing the skull (away from the cortical surface) for biosignal amplifier ground and reference. The electrodes can be as small as 50 microns with spacing of 0.5 millimeters.

BCI 100 also includes signal acquisition circuitry 106 that receives the raw signal from electrode array 104. Signal acquisition circuitry 106 includes, for example, a multiplexer, an amplifier, a filter, an analog-to-digital (A/D) converter, a transceiver, and a power supply (none shown in FIG. 1). An exemplary biosignal amplifier records ECoG signals and microphone data at a sampling frequency of 1.2 kilohertz and 24-bit resolution. Moreover, microphone signals used ground and references electrically isolated from the ECoG signals in order to prevent interference. An exemplary filter is a digital band pass filter that operates between approximately 0.1 Hz and 500 Hz. Signal acquisition circuitry 106 receives the raw signal from electrode array 104 and generates a transmission signal for use in determining an intended action by the subject. In one embodiment, signal acquisition circuitry 106 is included with electrode array 104 in a single fully-implantable housing. In another embodiment, signal acquisition circuitry 106 is remotely located from electrode array 104. In such an embodiment, electrode array 104 transmits the brain signals to signal acquisition circuitry 106 via a wired connection or wirelessly. Accordingly, in such an embodiment, electrode array 104 includes a transmitter (not shown in FIG. 1) that enables communication between electrode array 104 and signal acquisition circuitry 106.

Moreover, BCI 100 includes signal analysis circuitry 108, such as a computer. Signal analysis circuitry 108 includes, for example, a memory area and a processor (neither shown in FIG. 1). Signal analysis circuitry 108 receives the transmission signal from signal acquisition circuitry 106, decodes the transmission signal, and generates a control signal for use in controlling a device, such as device 110. For example, signal analysis circuitry 108 decodes the transmission signal, extracts features from the transmission signal, applies a translation algorithm to the features, and generates the control signal for controlling device 110. In some embodiments, the memory area includes computer-executable program modules or components (not shown in FIG. 1) that include computer-executable components. One exemplary component includes instructions for synchronizing stimuli presentation and ECoG and microphone signal recording. For example, stimulus periods of approximately four seconds are interleaved between 533 millisecond (ms) intertrial intervals (ITI), and visual stimuli is displayed for the entire stimulus period on a display (not shown). In addition, auditory stimuli are presented through headphones with an average duration of approximately 531 ms. In some embodiments, stimuli for both tasks are selected from a list of 36 monosyllabic English language words.

Another exemplary component includes instructions for calculating autoregressive power spectral density (PSD) estimates using, for example, the Yule-Walker method and a preselected model order that balances PSD smoothness with an ability to precisely detect known sinusoidal noise peaks from environmental noise. Another exemplary component includes instructions for generating cortical activation plots, such as those described below, and a percentage of patients with significant activations by frequency using significant R² values at each frequency bin. Yet another exemplary component includes instructions for detecting activation flips using normalized spectra, which facilitates removing non-stationary changes in brain state and environmental noise that occur on short, such as less than four seconds, time scales. Moreover, such instructions facilitate equalizing scales for power increases and decreases, and providing a basis of comparison of power changes.

In some embodiments, signal analysis circuitry 108 is included with electrode array 104 and/or signal acquisition circuitry 106 in a single housing. In other embodiments, signal analysis circuitry 108 is located remote from electrode array 104 and/or signal acquisition circuitry 106. Moreover, signal analysis circuitry 108 communicates with signal acquisition circuitry 106 via a wired connection or wirelessly.

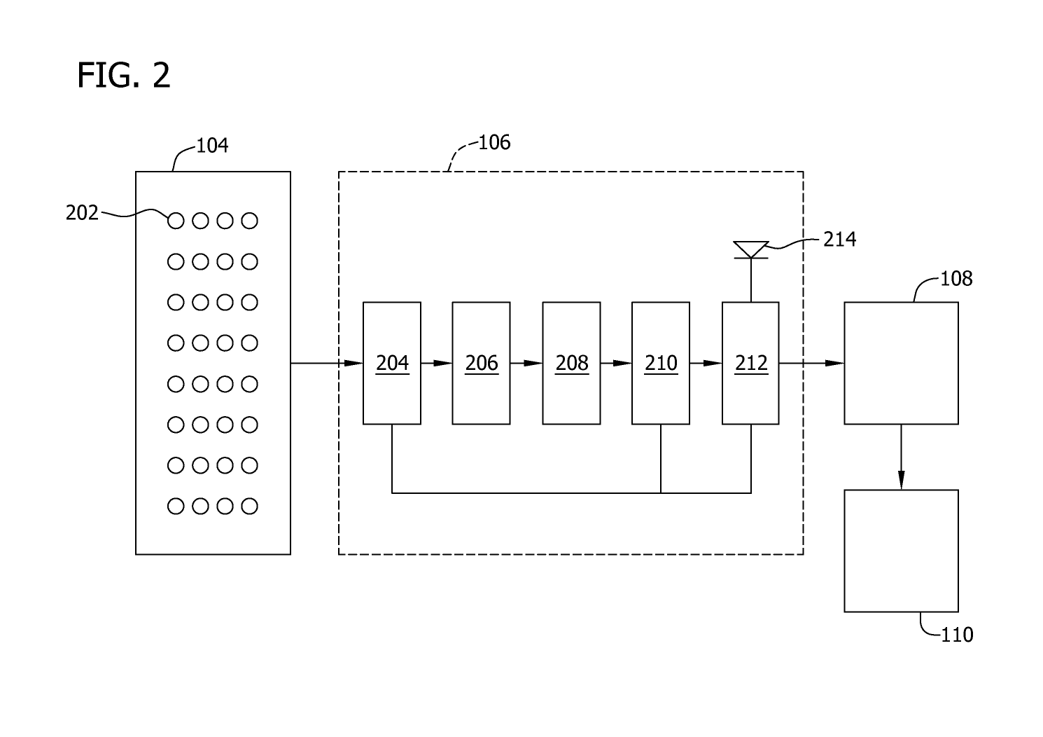

FIG. 2 is a block diagram of signal acquisition circuitry 106. As shown in FIG. 2, signal acquisition circuitry 106 is adapted for communication with electrode array 104 to convert analog brain signals acquired by electrodes 202 to a transmission signal representative of the brain signals. The brain signals are multiplexed, amplified, filtered, and converted from analog to digital. Moreover, in one embodiment, each of the components described below of signal acquisition circuitry 106 are mounted on a flexible substrate, such as a circuit board. Furthermore, in some embodiments, one or more of the components described below are combined such that a single chip provides the functionality described below.

Signal acquisition circuitry 106 includes a multiplexer 204 that receives the brain signals from electrode array 104 via a plurality of channels. For example, in one embodiment, electrode array 104 acquires sixteen channels of analog data. Multiplexer 204 receives the sixteen channels and multiplexes them into a single channel at a desired frequency, such as 8 kHz. In one embodiment, multiplexer 204 switches through each channel and holds the received channel for a selected length of time. Multiplexer 204 holds a signal from a single channel by multiplying the channel by a constant voltage pulse. During a transition time, multiplexer 204 switches to a next channel and adds the multiplied value to the single output channel.

Moreover, signal acquisition circuitry 106 includes an amplifier 206 coupled to multiplexer 204, and a low-pass filter 208 coupled to amplifier 206. Filter 208 removes high-frequency distortions from the amplified signal and prevents aliasing before the signal is converted from analog to digital. An analog-to-digital (A/D) converter 210 synchronizes with multiplexer 204 and with a clock signal supplied by a transmitter 212. In addition, A/D converter 210 addresses each channel within the signal to localize portions of the signal to respective electrodes 202. A/D converter 210 outputs a digital transmission signal to transmitter 212, which is transmitted to signal analysis circuitry 108 via an antenna 214. An exemplary transmitter 212 is a Bluetooth® transmitter (Bluetooth® is a registered trademark of Bluetooth Sig, Inc., Bellevue, Wash., USA). However, any suitable wireless or wired transmitter may be used.

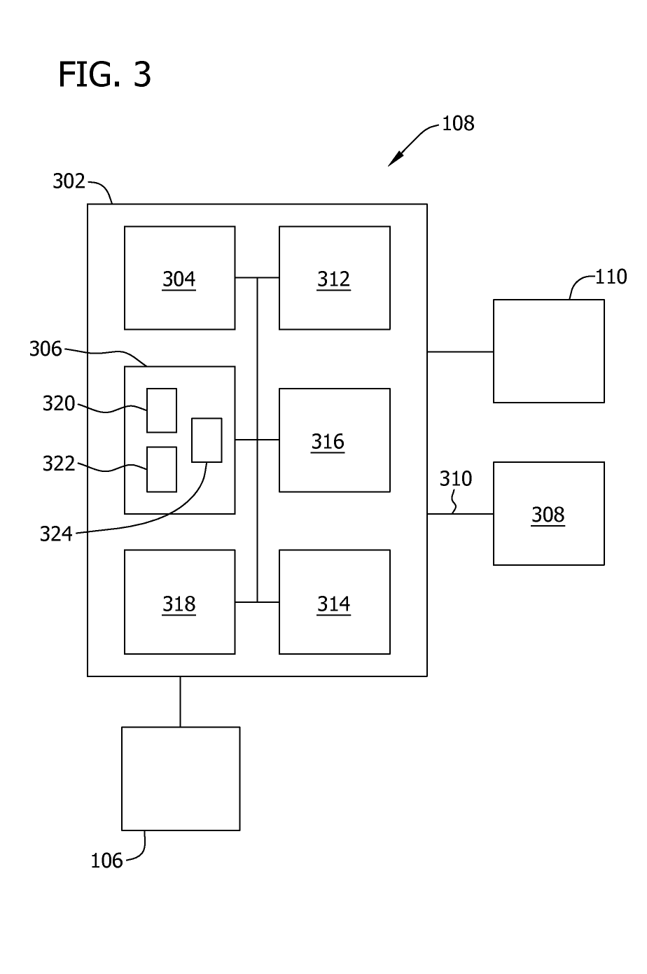

FIG. 3 is a block diagram of signal analysis circuitry 108. In the example of FIG. 3, signal analysis circuitry 108 is embodied as a computer 302. However, any suitable form may be used, such as a Personal Digital Assistant (PDA), a Smartphone, or any other suitably equipped communication device. As shown in FIG. 3, computer 302 includes a processor 304 and a memory area 306 coupled to processor 304. In some embodiments, computer 302 includes multiple processors 304 and/or multiple memory areas 306. Moreover, memory area 306 may be embodied as any suitable memory device or application including, but not limited to, a database, a hard disk device, a solid state device, or any other device suitable for storing data as described herein. Furthermore, memory area 306 is located within computer 302. Alternatively, memory area 306 may include any memory area internal to, external to, or accessible by computer 302. Further, memory area 306 or any of the data stored thereon may be associated with any server or other computer, local or remote from computer 302 (e.g., a second computer 308 coupled to computer 302 via a network 310).

Computer 302 includes a display device 312, a secondary storage device 314 such as a writable or re-writable optical disk, and input/output devices 316 such as a keyboard, a mouse, a digitizer, and/or a speech processing unit. In addition, computer 302 includes a transceiver 318 that receives the digital transmission signal from transmitter 212 (shown in FIG. 2) and transmits a control signal to device 110.

In some embodiments, memory area 306 includes one or more computer-readable storage media having computer-executable components. For example, memory area 306 includes a communication component 320 that causes processor 304 to receive the digital transmission signal from signal acquisition circuitry 106 via transceiver 318, a signal analysis component 322 that converts the received signal into a control signal for use in controlling device 110 according to an intended action by the subject, and a control component 324 that uses the control signal to control device 110.

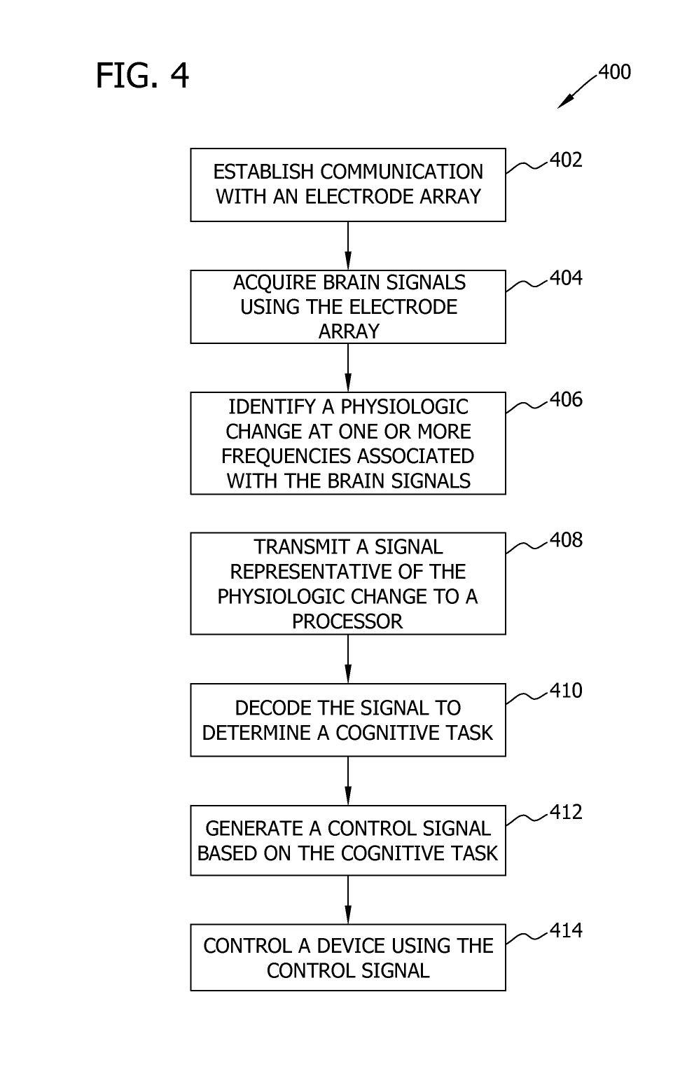

FIG. 4 is a flowchart 400 that illustrates an exemplary method of associating the one or more of a plurality of frequencies with a cognitive task. Initially, communication is established 402 with electrode array 104 (shown in FIG. 1) implanted beneath the scalp of a subject. Communication may be established via a wired or wireless connection between electrode array 104 and signal acquisition circuitry 106 (shown in FIG. 1). Electrode array 104 acquires 404 brain signals at a plurality of frequencies via a plurality of electrodes 202 (shown in FIG. 2) at a single portion of the brain or at multiple portions of the brain simultaneously.

Signal acquisition circuitry 106 receives the brain signals and identifies 406 a physiologic change at one or more of the frequencies. For example, signal acquisition circuitry 106 processes the brain signals to generate a transmission signal, using multiplexer 204, amplifier 206, low-pass filter 208, and analog-to-digital converter 210 (each shown in FIG. 2). Signal acquisition circuitry 106 then transmits 408 the transmission signal representative of the physiologic change to signal analysis circuitry 108 (shown in FIG. 1) via, for example, transmitter 212 (shown in FIG. 2). Exemplary physiologic changes that may be detected and used to determine a desired task include, but are not limited to, an amplitude change, a change in phase, a change in phase power coupling, and/or a change in event related potential.

Signal analysis circuitry 108 receives the transmission signal via transceiver 318 (shown in FIG. 3), and decodes 410 the transmission signal using processor 304 (shown in FIG. 3). In some embodiments, signal analysis circuitry 108 stores the decoded transmission signal in memory area 306 or in secondary storage 314 (both shown in FIG. 3). Processor 304 determines an intended cognitive task based on the physiologic change within the brain signals, and generates 412 a control signal representative of the cognitive task. Signal analysis circuitry 106 then controls 414 device 110 using the control signal. Notably, the cognitive task associated with one or more physiologic changes may change over time. Accordingly, in some embodiments, signal analysis circuitry 108 is capable of re-learning the frequency signatures that are associated with a cognitive task. For example, signal analysis circuitry 108 detects when the frequency signatures of the tasks change as a person ages, develops, has medical problems, takes certain drugs, and the like, and stores the changed frequency signature in memory area 306. Furthermore, in some embodiments, signal analysis circuitry 108 detects abnormal brain activity by sensing unexpected frequency signatures for the cognitive tasks. In such an embodiment, signal analysis circuitry 108 is capable of detecting early dementia, Alzheimer's, seizures, epilepsy, stroke, etc.

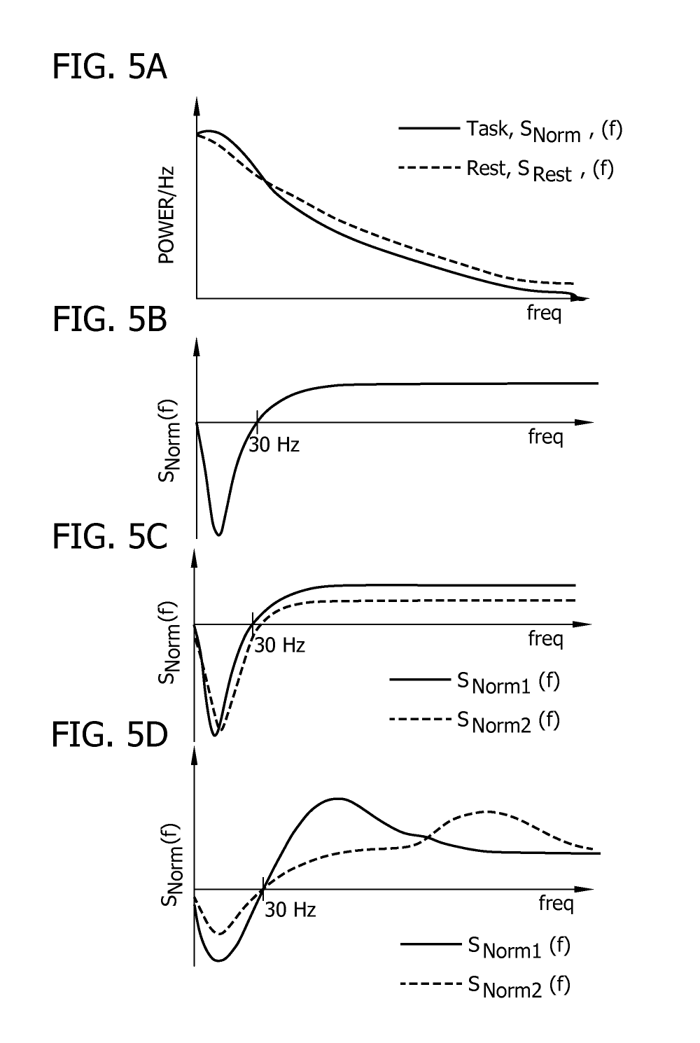

FIGS. 5A-5D are graphs that illustrate test results of seven right-handed subjects that clinically required the placement of electrode arrays 104 (shown in FIG. 1) over the surface of their left frontal and/or temporal cortex. Each subject performed two simple word repetition tasks cued with either auditory stimuli (i.e., the word has initially heard) or visual stimuli (i.e., the word was initially read). Spectral changes were assessed across multiple trials during the stimuli, including preparation to speak and the actual speaking, within the subjects and across subjects. As shown in FIGS. 5A-5D, ECoG signals contain non-uniform and narrowband power changes between 30 Hz and 530 Hz. For example, FIG. 5A illustrates a typical set of spectral densities where the solid line represents a frequency response to a task under observation (S_{Norm1}(f)) and the dashed line represents a frequency response during a intertrial interval that is the basis for comparison (S_{Rest}(f)). FIG. 5B illustrates a normalized power spectrum of the S_{Norm1}(f) response. The response may also be shown in equation form, as shown in Equation (1) below.

S_{Norm1}(f)=log(S_{Task1}(f))−log(S_{Rest}(f)) Eq. (1)

FIG. 5C is a graph of schematic normalized spectra to illustrate the idea that high frequency power change is uniform in nature. In addition, FIG. 5C illustrates that low frequencies, such as less than 30 Hz, tend to show power decreases for cognitive tasks while high frequencies show power increases. FIG. 5D is a graph of schematic normalized spectra to illustrate the idea that high frequency power change is non-uniform. Moreover, FIG. 5D shows that both spectra include power changes in narrow bands that may be used to distinguish one cognitive task from another.

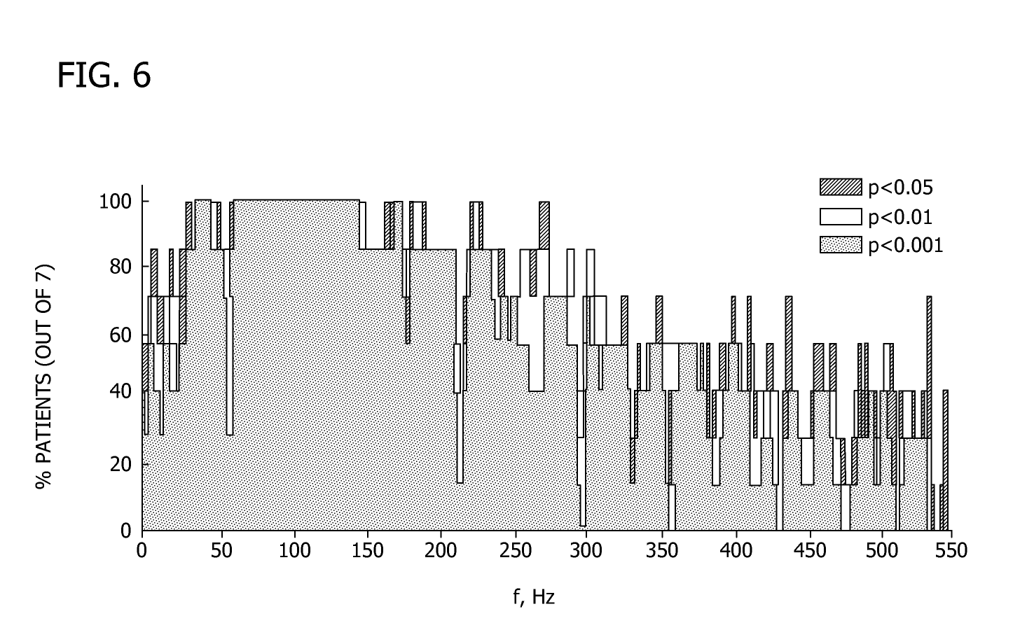

FIG. 6 is a graph that illustrates a percentage of the seven subjects that exhibited significant power change by frequency. More specifically, FIG. 6 is a graph that illustrates a percentage of subjects that exhibited statistically significant power changes by frequency. Table 1 below illustrates the trial data.

TABLE 1
Trial Data.

| | | | | | | Number |
|---|---|---|---|---|---|---|
| | | | | | | of |
| | | | | | | Trials |
| | Age/ | | Cognitive | Grid | Epileptic | Per |
| Subject | Sex | Hand | Capacity | Location | Focus | Task |
| 1 | 15/M | R | Normal | L-F | Left Frontal | 216 |
| | | | | | Supplementary | |
| | | | | | Motor Area | |
| 2 | 15/F | R | Normal | L-F/P | Left Temporal | 72 |
| 3 | 44/M | R | Avg to | L-F | Left Orbito- | 72 |
| | | | High Avg | | Frontal | |
| 4 | 27/M | R | Low | L-F/P | Right Mesial | 180 |
| | | | Average | | Parietal | |
| | | | (FSIQ 89, | | | |
| | | | VIQ 86, | | | |
| | | | PIQ 96) | | | |
| 5 | 58/F | R | High Avg | L-F/P | Superior | 216 |
| | | | (FSIQ | | Frontal Gyms | |
| | | | 116) | | | |
| 6 | 49/F | R | Avg | L-F/T | Anterior | 216 |
| | | | (FSIQ | | Temporal | |
| | | | 100) | | | |
| 7 | 42/F | R | Low Avg | L-F/T/P | Anterior | 109 |
| | | | (FSIQ 81) | | Temporal/ | |
| | | | | | Amygdala/ | |
| | | | | | Hippocampus | |

As shown in FIG. 6, across the subject population, consistent task-evoked high frequency power changes were present at frequencies as high as 546 Hz. Statistically significant coefficient of determination (R²) values counted across cognitive tasks and electrodes indicate that five of the seven subjects exhibited significant evoked power changes up to 534 Hz (p<0.05). With the most stringent statistical test (p<0.001), two of the seven subjects exhibited significant activations as high as 532 Hz. As will be described in greater detail below, these activations were neither uniform nor broadband in nature.

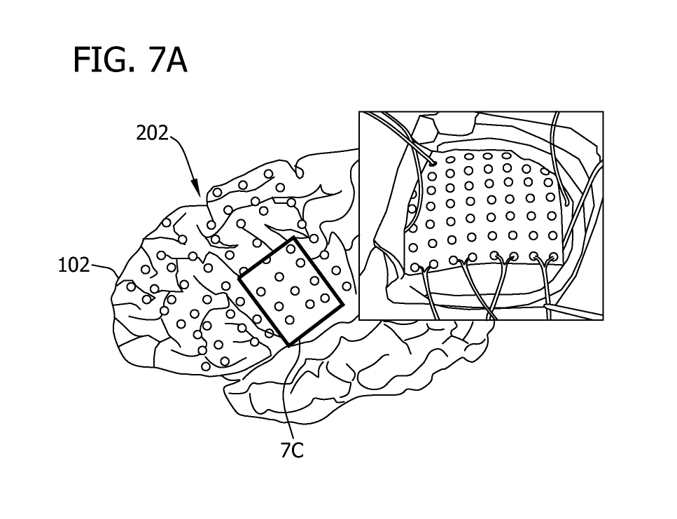

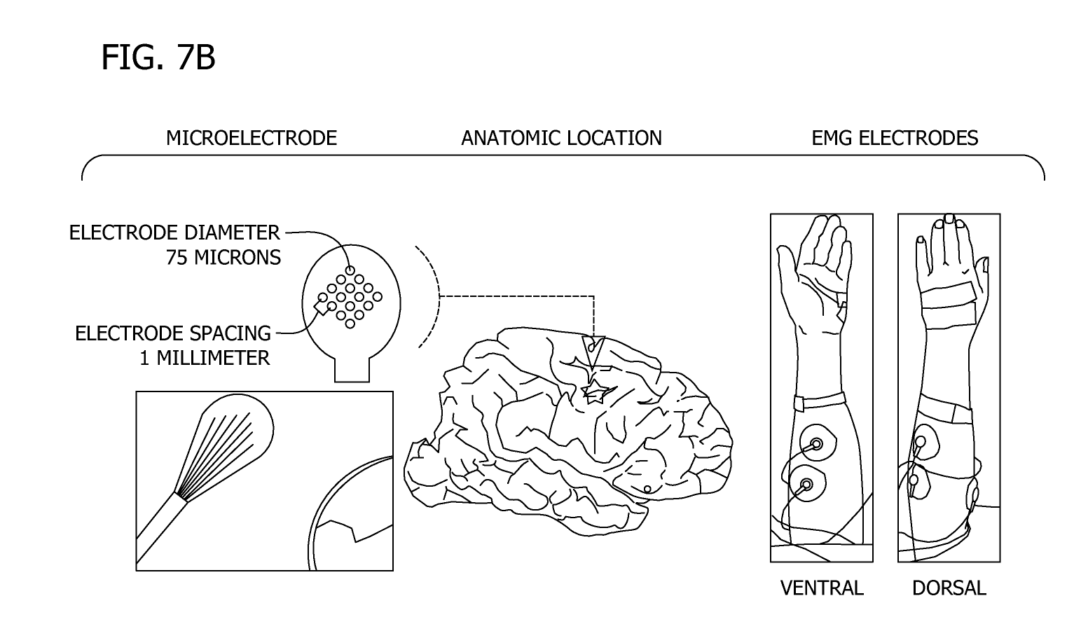

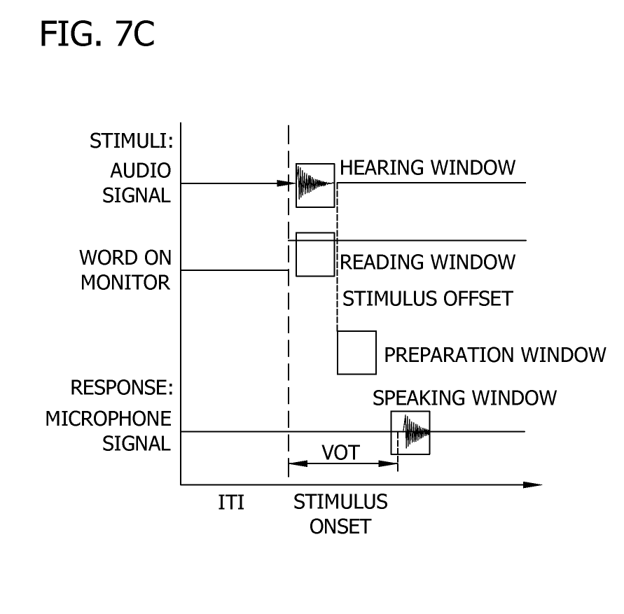

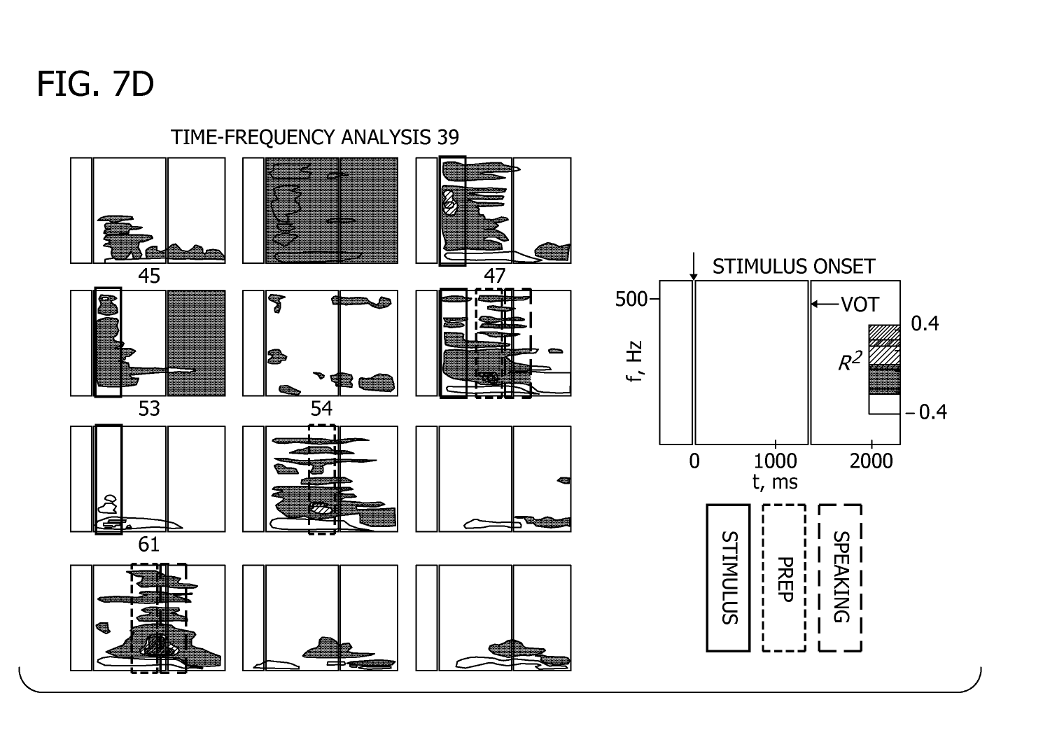

FIGS. 7A-7C illustrate an exemplary experimental setup as defined for Table 1 above. Specifically, FIG. 7A is a view of implanted ECoG electrodes 202 and corresponding localization on a brain model. FIG. 7B is a view of a microgrid that may be used to acquire brain signals from the subject. A microgrid is the size of a single electrode, but includes 75 micron electrodes spaced approximately 1 mm apart. Microgrids enable minimally invasive implants. FIG. 7C is a graph showing timing of two different experimental paradigms. Single word stimuli were presented either aurally or visually. Analysis windows for hearing and reading are cued to stimulus presentation, preparation analysis windows are cued to stimulus effect, and windows for speaking are cued to voice onset detected from a microphone signal. FIG. 7D shows exemplary time-frequency plots for the auditory repeat program shown in FIG. 7C. The plots of FIG. 7D exhibit a significant (p<0.001) R² values for twelve electrodes. Six electrodes of interest are numbered in FIG. 7D and correspond to the filled electrodes shown in FIG. 7A. The rectangles highlight notional analysis windows with non-uniform change patterns.

As another example, ECoG signals were recorded as the subjects performed a modified center out task using a hand held joystick. Delay periods were added to the task in order to be able see target encoding without movement confounding this data. This was done to more closely match the delay match to sample task from the traditional monkey paradigms. There were 5 different important periods to the task: baseline (300 ms), encoding (500 ms), delay (300, 400, or 500 ms), movement, and holding (300 ms). A baseline was collected prior to display of the target, by changing the color of the “correct” target. A delay period followed the target encoding period, where the subject had to hold the target in memory. At the end of the delay period, a ring and circle in the center would disappear as a go signal for the subject to use a joystick to move the cursor to the appropriate target (i.e. movement period). Once the subject reached the target they held the cursor on the target for a period of time. The task had 8 targets placed radially and equidistant (45 degrees apart) around a center starting point to be of maximum diameter on the 15 inch Dell LCD display. The targets were presented in a randomized order. All subjects were presented each of eight targets five times over two runs for a total of eighty movements for each subject. Any incorrect trials were not repeated and removed from further analysis.

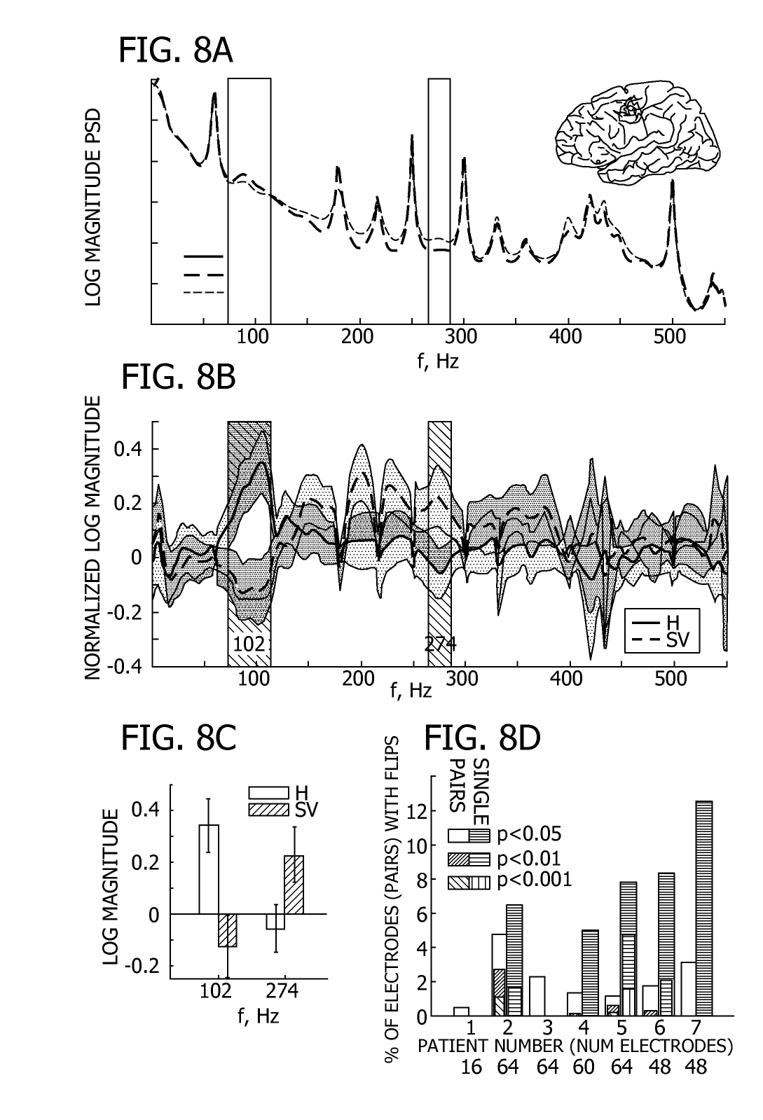

FIGS. 8A-8D illustrate a means of quantifying the non-uniform and narrowband nature of the evoked spectra, which is referred to herein as “activation flips.” Specifically, FIG. 8A is a graph that shows exemplary mean power spectral densities for rest and two cognitive tasks, where H is a hearing action and SV is a speaking task after a visual cue. As shown in FIG. 8A, there are multiple narrow bands where reversals in power change magnitude occur. FIG. 8B is a graph that shows mean normalized spectra as calculated from FIG. 8A with a 99% confidence interval, and that shows two different activation patterns. For example, the bands centered at 102 Hz and at 274 Hz have the largest magnitude of reversal in power change magnitude, which demonstrates an activation flip. FIG. 8C is a graph that further illustrates the activation flips shown in FIG. 8B, and FIG. 8D is a graph that shows a percentage of electrodes or electrode pairs that exhibited activation flips by subject and p-value, where the frequency bands are between 60 Hz and 550 Hz.

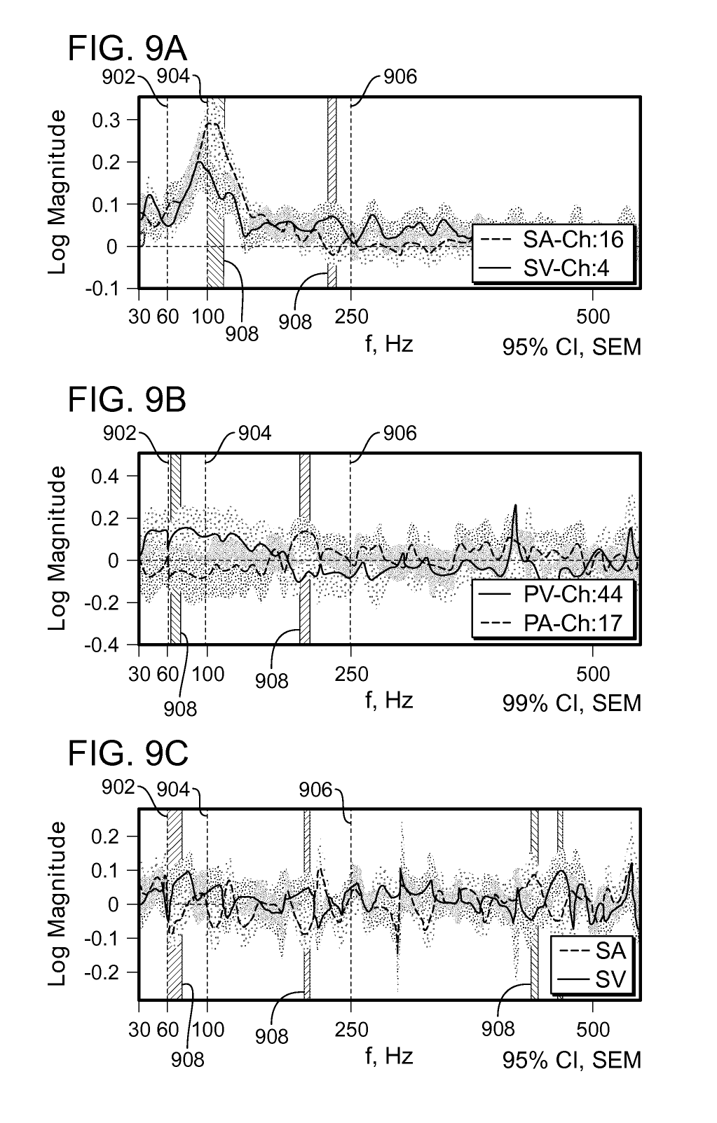

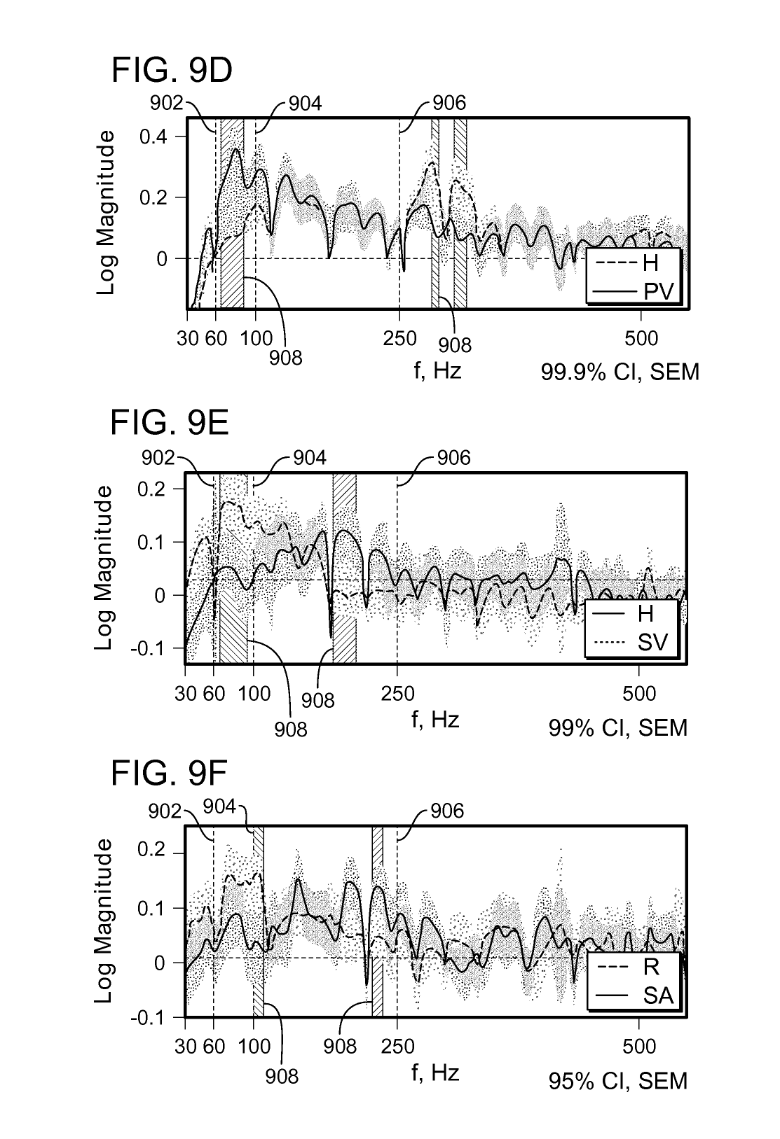

FIGS. 9A-9F are graphs showing individual subject normalized spectral responses that illustrate activation flips for a subset of the seven subjects. FIGS. 9A and 9C correspond to subjects that did not exhibit single electrode activation flips. As such, FIGS. 9A and 9C illustrate the use of two different electrodes. Markers 902, 904, and 906 at 60 Hz, 100 Hz, and 250 Hz, respectively, outline typical gamma analysis bands. Bands 908 highlight areas were confidence intervals to not overlap.

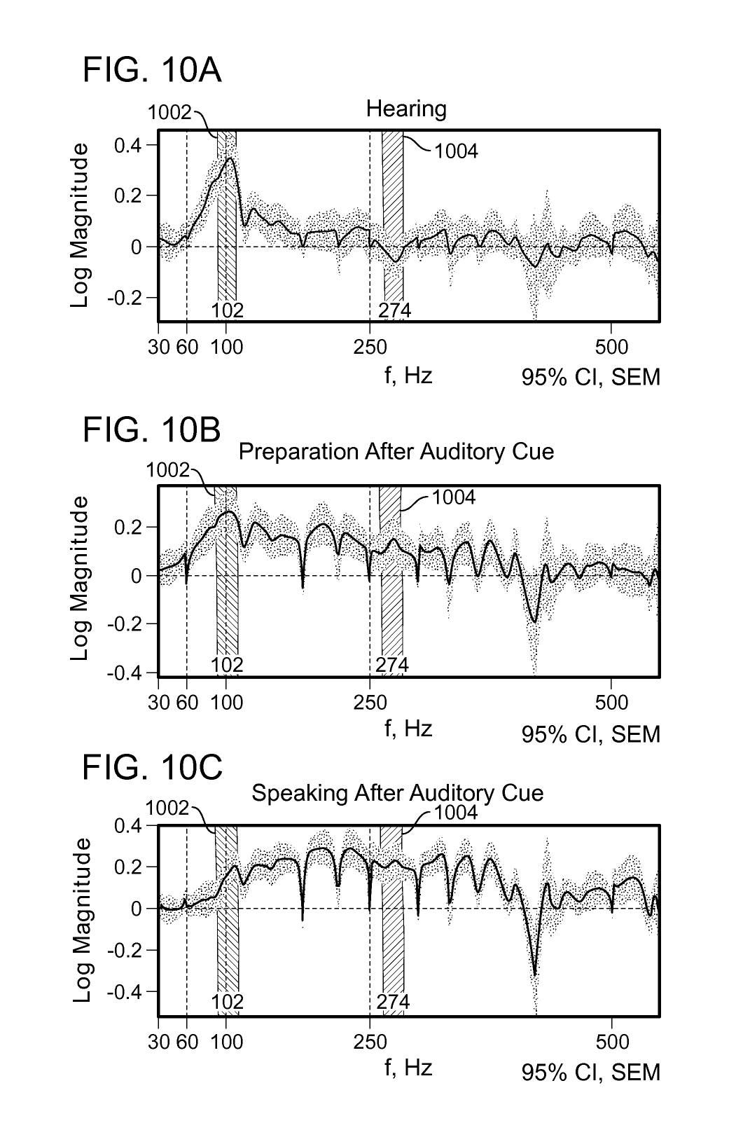

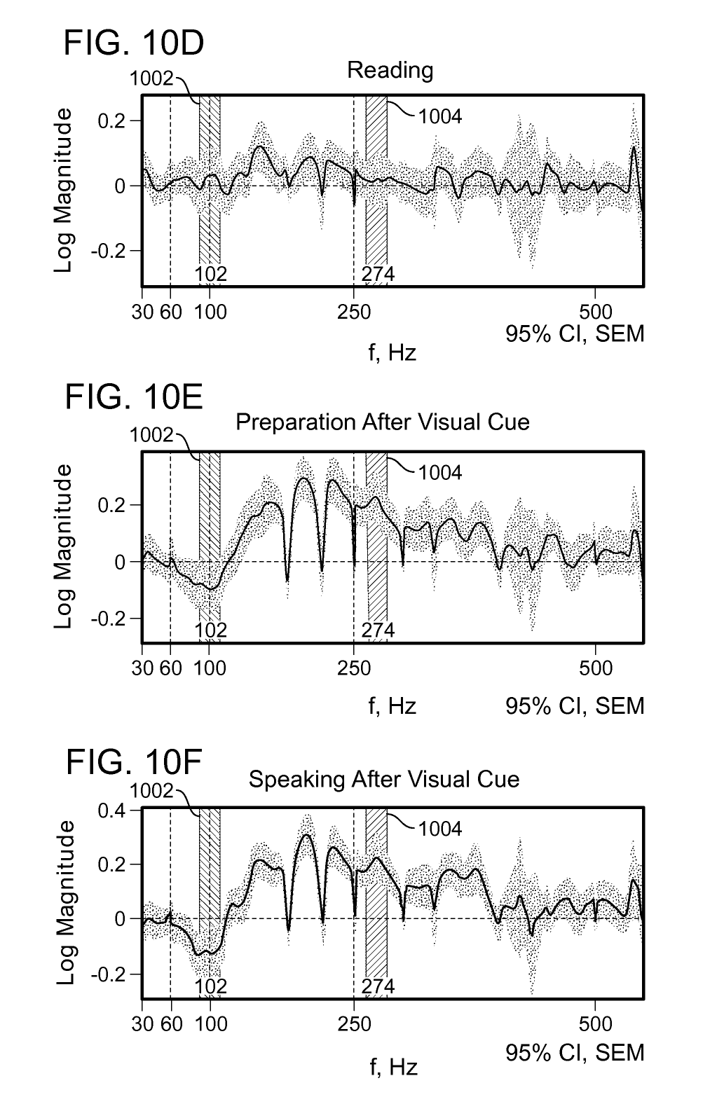

Each of the seven subjects had electrodes 202 (shown in FIG. 2) with evoked spectra that reveal power changes concentrated in specific frequency bands. Such narrowband activations are visible in a normalized log magnitude spectra, as shown in FIG. 8B. The log magnitude spectra of the evoked power changes for all subjects and activities were non-uniform, as shown in FIGS. 10A-10F, and revealed statistically significant power changes in different bands and with different magnitudes.

Referring again to FIGS. 8B and 8C, in one frequency band, the magnitude of the normalized power change for task A (e.g., hearing) is larger than that of task B (e.g., speaking after a visual cue). At a second frequency band, the magnitudes of the normalized power change reverse between these tasks (i.e., task B evoked a larger magnitude power change than task A). In order to count an activation flip, the active bands between the compared conditions rely on non-overlapping confidence intervals (standard error) for at least 6 Hz in each frequency band.

FIGS. 10A-10F are graphs that show normalized spectra for a single channel across all six cognitive tasks for the same subject shown in FIG. 8C. Frequency bands 1002 and 1004 centered at approximately 102 Hz and 274 Hz, respectively, illustrate the activation flip between hearing and speaking after a visual cue. The normalized spectra of FIGS. 10A-10F illustrate that the two frequency bands of interest 1002 and 1004 activate independently and do not flip as an artifact of signal processing. For example, while speaking after an auditory cue, both bands 1002 and 1004 exhibit significant power increases. As another example, while reading, neither band 1002 and 1004 is statistically different than the rest, but a band centered at approximately 150 Hz exhibits a significant power increase.

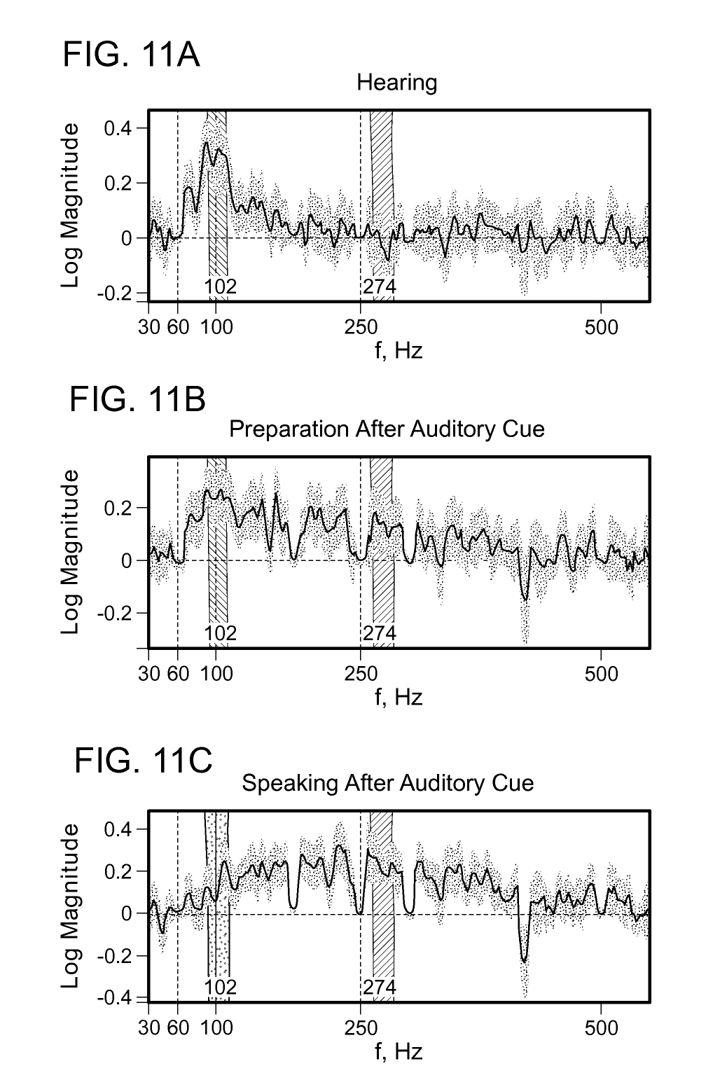

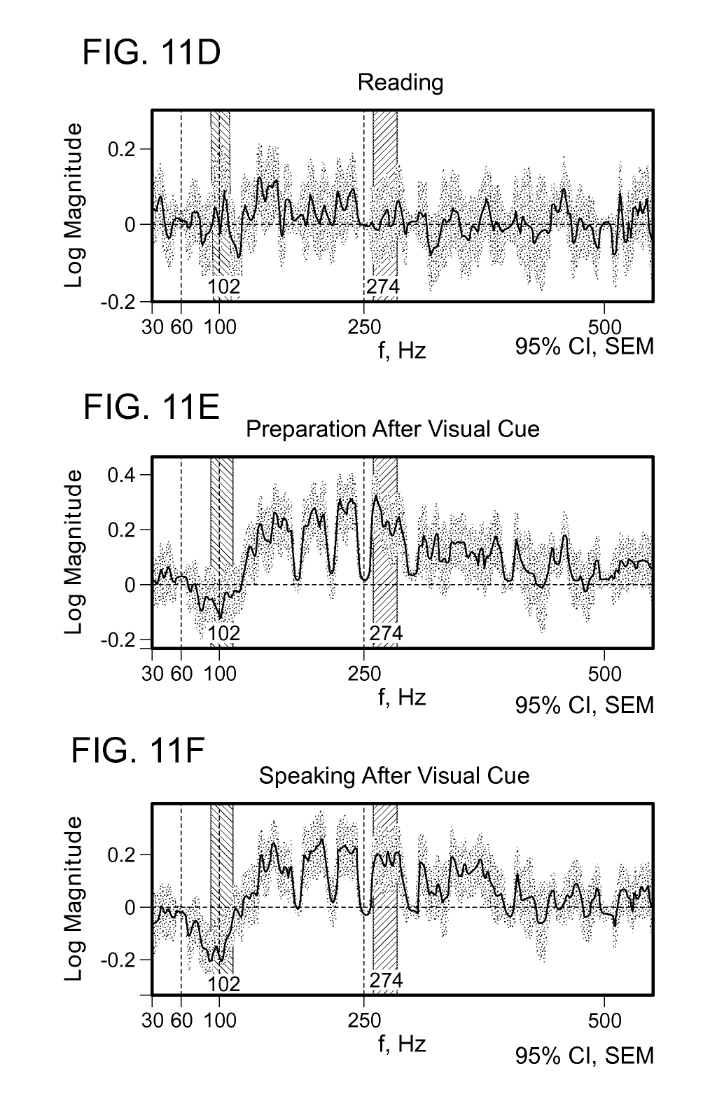

FIGS. 11A-11F are graphs that show normalized spectra computed using Fast Fourier Transforms (FFT) instead of the autoregressive method used to generate the spectra of FIGS. 10A-10F. Each PSD was computed using a 512-point FFT with hamming windows. Notably, the normalized spectra for each cognitive task in FIGS. 10A-10F and FIGS. 11A-11F are similar. Moreover, the autoregressive model used in FIGS. 10A-10F did not introduce narrow band, non-uniform high frequency power changes.

Referring again to FIG. 8D, multiple activation flips were identified for each subject. Specifically, FIG. 8D shows a number of activation flips for each subject. The number of activation flips between electrode pairs is normalized by the number of possible pairs and plotted as a percentage. As can be seen in FIG. 8D, each subject exhibited significant (p<0.05) pair-wise activation flips. The number of activation flips for each subject depended on the strength of statistical test. However, five of the seven subjects exhibited single electrode activation flips that were significant for p<0.05 and one of the seven subjects exhibited significant single electrode activation flips at p<0.001.

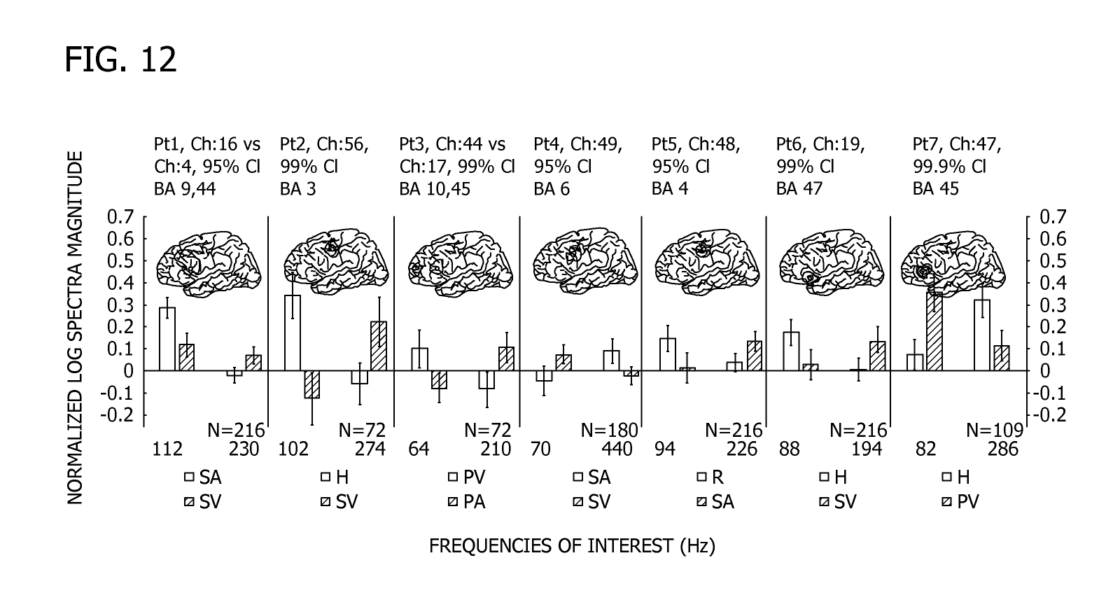

FIG. 12 is a set of graphs that show activation flips for the seven subjects. For the two subjects without activation flips from single electrodes, examples were selected from two different electrodes. In general, it is unlikely that asynchronous neuronal firing activity, which may result in uniform broadband power changes, caused the activation flips shown in FIG. 12. Rather, it should be understood that the narrowband, high frequency, power change reversals illustrated in FIG. 12 show that ECoG is capable of capturing synchronous oscillatory activity at different frequencies from within the same cortical population.

Evaluating cortical activations over a broad range of frequencies shows that power changes occur non-uniformly even within small populations. Three cortical regions—the left sensorimotor cortex (Broadmann Areas (BA) 1-4), Broca's area (BA 44 and 45), and the left posterior superior temporal gyrus (STG, BA 42)—have all been implicated in functional imaging studies using similar language tasks. For each combination of cortical region and cognitive task, bar plots show that the percentage of electrodes in each region with statistically significant R² values (p<0.001, Bonferroni corrected for 50 comparisons) at each frequency. If cortical power changes occurred uniformly across frequencies, as shown in FIG. 5C, the cortical activation plots would be flat. However, as shown below, that is not the outcome of the experiment described herein.

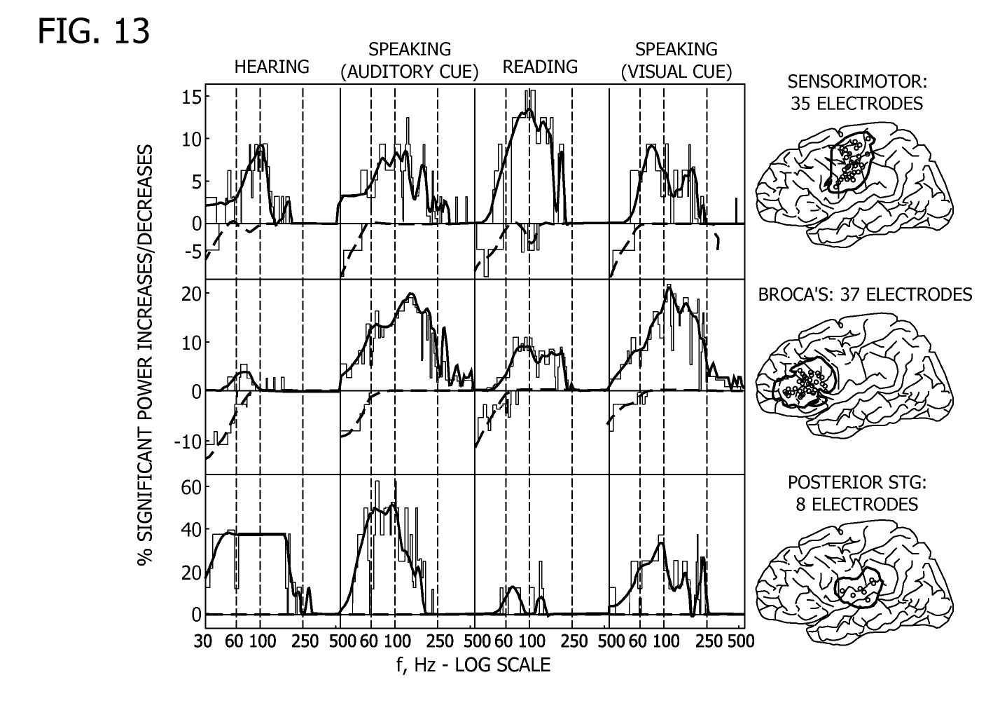

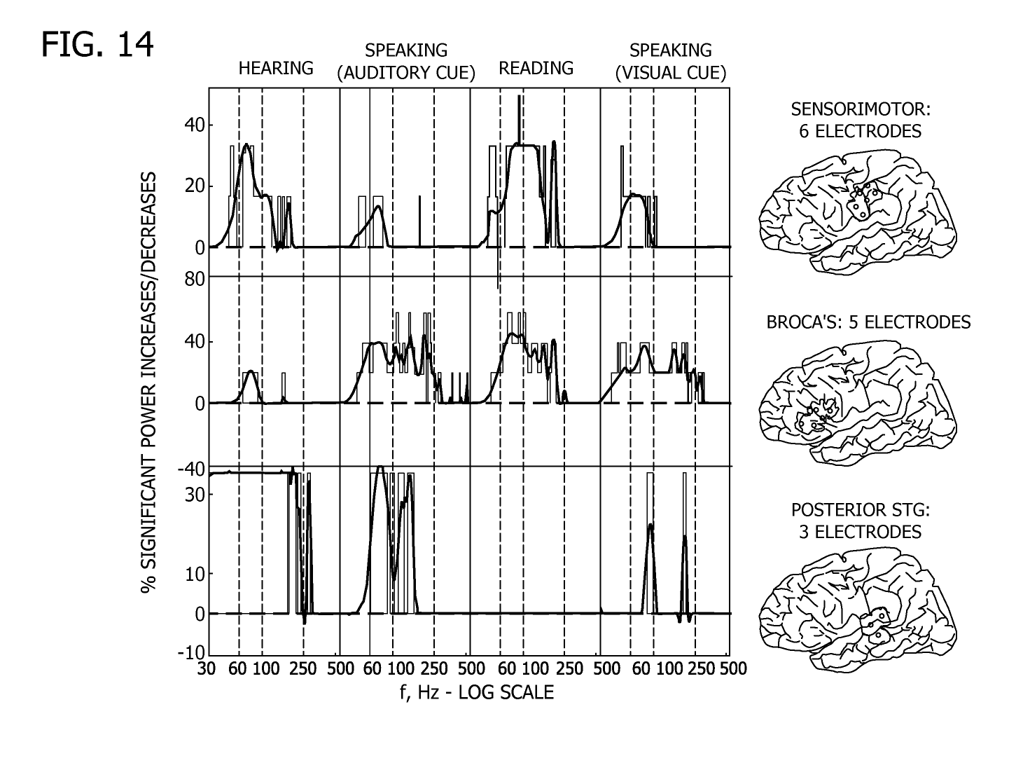

FIGS. 13 and 14 illustrate four trends in the consolidated cortical activation plots that the support the ideas that high frequencies activate non-uniformly and that activations depend on both cognitive task and anatomy. Specifically, FIG. 13 shows consolidated cortical activation plots for seven subjects, and FIG. 14 shows cortical activation plots for a single subject. In both FIGS. 13 and 14, positive numbers indicate a percentage of electrodes with statistically significant (p<0.001) power increases, negative numbers correspond to power decreases, rows of activation plots correspond to cortical regions, and columns correspond to cognitive tasks. The frequency is plotted on a logarithmic scale between approximately 30 Hz and 550 Hz to facilitate visualizing power changes at lower frequencies. Markers positioned at approximately 60 Hz, 100 Hz, and 250 Hz indicate typical gamma or high gamma analysis boundaries. Notably, multiple peaks per plot, shifts in percentage of cortex active frequency bands, and changes in active bandwidth within cortical populations are all evidence of non-uniform power changes in these high frequency bands.

In a first trend, many single activation plots exhibit multiple peaks, such as sensorimotor-speaking after auditory cue, Broca's-speaking after visual cue, and posterior STG-reading. These are an indication of statistically significant narrowband power changes in different frequency band during the same task and within the same cortical area. Second, within cortical regions, cognitive tasks have either distinct active bandwidths or changing cortical representations within similar active bandwidths, but are separable by the different proportions of cortex engaged across the range of active frequencies (i.e., speaking after auditory cue appears more unimodal, while speaking after visual cue appears bimodal). This second trend shows that the cortical region activates at different frequencies in a task-dependent manner. Third, for any given cognitive task, there is generally a variation in the active bandwidth between the three cortical regions, such that there does not appear to be a unified activation bandwidth across cortex for a specific cognitive task.

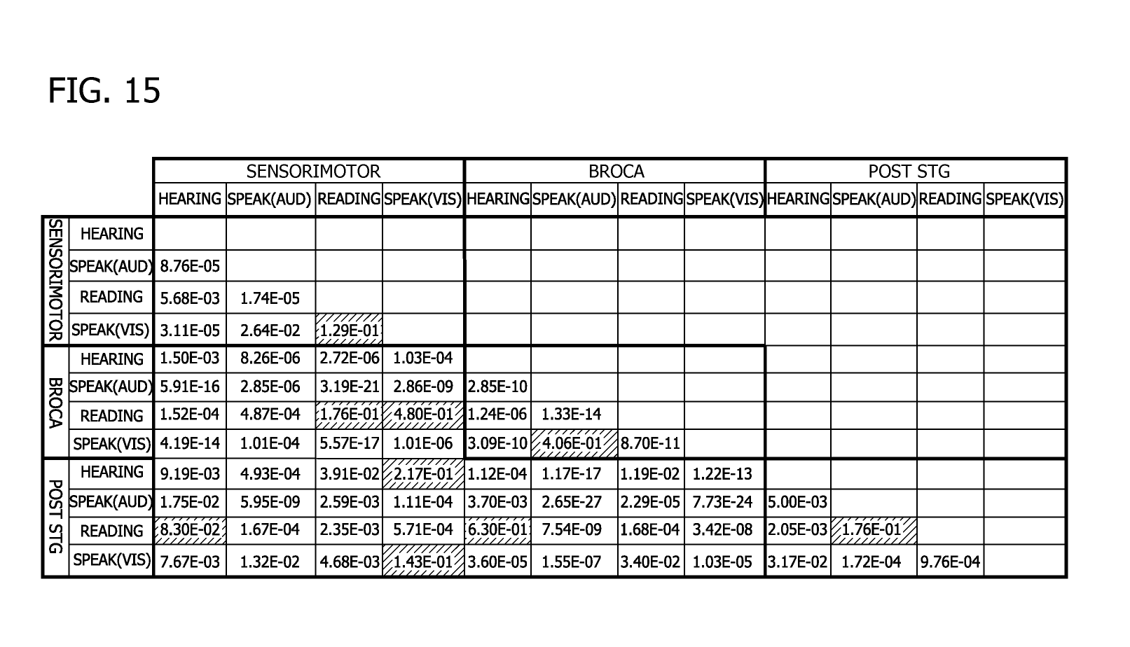

A quantitative measure of the second and third trends described above is shown in FIG. 15, which is a tabular set of results of Kolmogorov-Smirnoff tests. For example, the values shown in FIG. 15 are results of statistical tests of a null hypothesis that the shapes of individual cortical activation plots are from the same distribution, wherein approximately 86% of cortical activation plot comparisons are statistically distinct. In FIG. 15, shaded blocks indicate that the null hypothesis may be rejected when p<0.05. Moreover, bold lines outline regions that have common comparisons. Notably, within each cortical region, only a single test of the null hypothesis was not rejected, which indicates that the cortical activations were statistically different at the p<0.05 level. For each cognitive task, at least two cortical regions have activation plots that are statistically different.

Referring again to FIGS. 13 and 14, a fourth trend is that, despite the distinct activation patterns for cognitive task and anatomic region, there are still generalized trends present between cortical regions. For example, there were no activations in the posterior STG above approximately 300 Hz in contrast to the sensorimotor cortex and the Broca's area, which both had activations as high as approximately 530 Hz. Although the two regions in the frontal cortex exhibited significant activations up to 530 Hz, Broca's area exhibited the most consistent activations at high frequencies, as shown in the two productive speech activities. Posterior STG electrodes indicated few or no high frequency power decreases. Both frontal areas exhibited power decreases in multiple frequency bands as high as approximately 122 Hz.

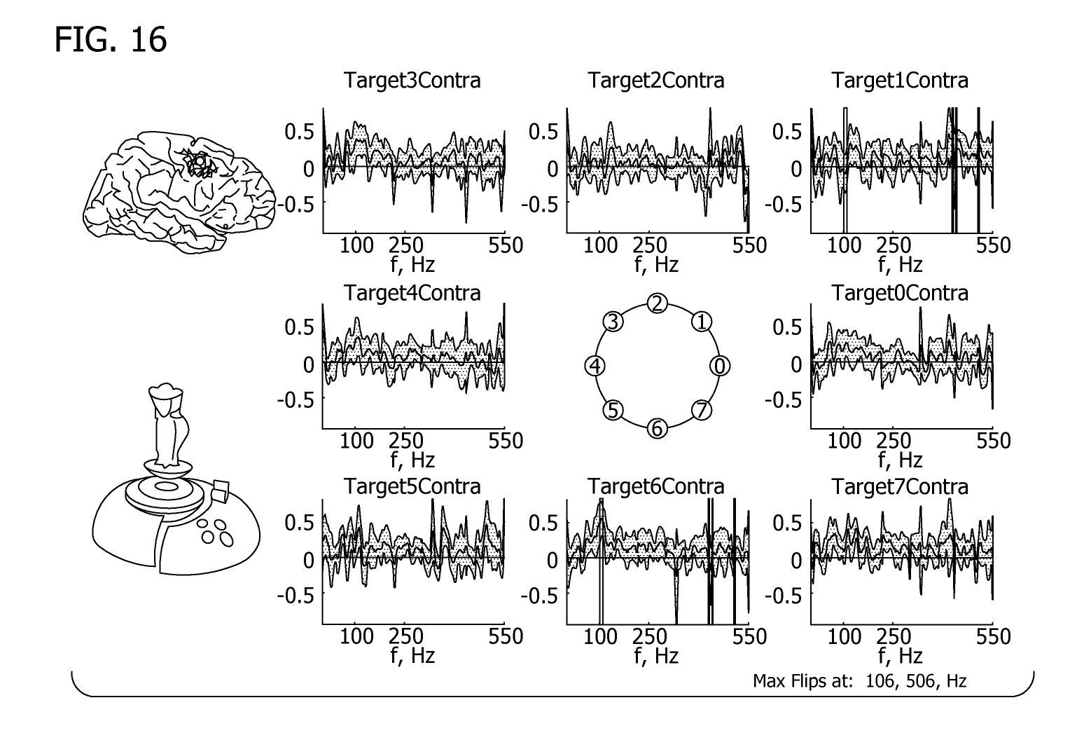

FIG. 16 is a set of normalized spectra that were defined while a subject performed a center joystick task. It can be seen that there are distinct frequency amplitude patterns for each of the specific directions. Thus, not only are specific spectral responses able to discern specific stages of a cognitive task as has been shown above. These spectral response can be utilized to distinguish specific elements of the cognitive intent. Namely, these specific spectral responses can not only define that a subject is intending a motor movement, but these specific spectral responses can define where the patient is intending to move (i.e. specific information on the cognitive intent versus a more general process occurrence). Accordingly, in an experiment where a subject uses the joystick to move a cursor from the center of the screen to one of six targets on the periphery of the screen, the ECoG signal may be recorded from a single area in premotor cortex (as indicated by the brain figure with the dot). The different normalized log spectra exhibit, via different physiologic changes at different frequencies of the ECoG signal, when the subject moved one particular direction. Accordingly, different stages of a task can be correlated as described in greater detail above (namely getting a cue to speak, preparing to speak, and actually speaking). Moreover, actual and specific aspects of the cognitive intention can be determined from the different normalized log spectra. Thus, it can be determined not only that the subject is moving, but also know where the subject is moving.

The systems, methods, and apparatus described herein facilitate capturing surface cortical potentials using ECoG, and having non-uniform, narrowband evoked power changes across frequencies from approximately 30 Hz to 530 Hz that depend on both cognitive task and anatomy. The power changes illustrated using activation flips and cortical activation plots are not caused by uniform power increases.

Known analyses have demonstrated that physiologically relevant cortical power changes may occur at various high frequencies. These oscillations have both normal and pathological sources. For example, the indiependence of power changes between a low gamma band, e.g., approximately 30 Hz to 60 Hz, and a high gamma band, e.g., approximately 60 Hz to 200 Hz, has been previously reported in humans using auditory stimuli with both active listening tasks and passive listening tasks. This distinction is confirmed by identifying twenty single electrode activation flips between approximately 30 Hz and 60 Hz and at frequencies above approximately 60 Hz. The activation flips shown in FIG. 8D, for example, further extend this subparcellation above 60 Hz. The low gamma oscillations are typically considered to be caused by alternating excitatory and inhibitory post-synaptic potentials. The physiological underpinnings of oscillations between 60 Hz and 200 Hz, however, are less clear. Studies of multi-unit recordings in non-human primates have shown correlations between local field potentials in the range of 60 Hz and 200 Hz and neuronal firing rates, but these results have not been correlated to surface cortical potentials. Higher frequency oscillations, for example, up to approximately 600 Hz, caused by peripheral nerve stimulation have been reported in non-human primate epidural and single unit recordings, and in human scalp EEG or MEG results. However, higher oscillatory frequencies between, for example, 200 Hz and 600 Hz, appear to be correlated to summation of action potential spiking. In addition tot eh natural physiological processes discussed above, evidence of high frequency “fast ripples” of less than approximately 500 Hz have been reported in human epileptic hippocampus. The strength of the statistical tests (p<0.001) described herein and used to correlate high frequency power changes with specific cognitive tasks indicates that the observed high frequency power changes are not spontaneous occurrences.

As described herein, the sensorimotor cortex exhibited strong activations in all four cognitive tasks, which supports the findings that the sensorimotor cortex is involved in phonetic encoding, formulation of motor articulatory plans, and other task-specific motor control activities. Broca's area also exhibited robust cortical activations during speaking tasks, moderate activations during reading tasks, and minimal activations during both hearing tasks. These activations are likely attributable to the grapho-phoneme conversion process during reading as well as “syllabification,” or a late pre-articulatory response, in preparation for speech that occasionally occurs during the late phase of hearing. The activations in the left posterior STG were strongest during hearing and speaking after an auditory cue, moderate during speaking after a visual cue, and minimal during reading tasks. Primary auditory perception, phonological processing, and self-monitoring are likely functions that cause activations during hearing and speaking tasks.

Exemplary embodiments of systems, methods, and apparatus for determining a cognitive task associated with one or more brain signals are described above in detail. The systems, methods, and apparatus are not limited to the specific embodiments described herein but, rather, operations of the methods and/or components of the system and/or apparatus may be utilized independently and separately from other operations and/or components described herein. Further, the described operations and/or components may also be defined in, or used in combination with, other systems, methods, and/or apparatus, and are not limited to practice with only the systems, methods, and storage media as described herein.

A computer, such as that described herein, includes at least one processor or processing unit and a system memory. The computer typically has at least some form of computer readable media. By way of example and not limitation, computer readable media include computer storage media and communication media. Computer storage media include volatile and nonvolatile, removable and non-removable media implemented in any method or technology for storage of information such as computer readable instructions, data structures, program modules, or other data. Communication media typically embody computer readable instructions, data structures, program modules, or other data in a modulated data signal such as a carrier wave or other transport mechanism and include any information delivery media. Those skilled in the art are familiar with the modulated data signal, which has one or more of its characteristics set or changed in such a manner as to encode information in the signal. Combinations of any of the above are also included within the scope of computer readable media.

Although the present disclosure is described in connection with an exemplary computer system environment, embodiments of the disclosure are operational with numerous other general purpose or special purpose computer system environments or configurations. The computer system environment is not intended to suggest any limitation as to the scope of use or functionality of any aspect of the disclosure. Moreover, the computer system environment should not be interpreted as having any dependency or requirement relating to any one or combination of components illustrated in the exemplary operating environment. Examples of well known computer systems, environments, and/or configurations that may be suitable for use with aspects of the disclosure include, but are not limited to, personal computers, server computers, hand-held or laptop devices, multiprocessor systems, microprocessor-based systems, set top boxes, programmable consumer electronics, mobile telephones, network PCs, minicomputers, mainframe computers, distributed computing environments that include any of the above systems or devices, and the like.

Embodiments of the disclosure may be described in the general context of computer-executable instructions, such as program components or modules, executed by one or more computers or other devices. Aspects of the disclosure may be implemented with any number and organization of components or modules. For example, aspects of the disclosure are not limited to the specific computer-executable instructions or the specific components or modules illustrated in the figures and described herein. Alternative embodiments of the disclosure may include different computer-executable instructions or components having more or less functionality than illustrated and described herein.

The order of execution or performance of the operations in the embodiments of the disclosure illustrated and described herein is not essential, unless otherwise specified. That is, the operations may be performed in any order, unless otherwise specified, and embodiments of the disclosure may include additional or fewer operations than those disclosed herein. For example, it is contemplated that executing or performing a particular operation before, contemporaneously with, or after another operation is within the scope of aspects of the disclosure.

In some embodiments, the term “processor” refers generally to any programmable system including systems and microcontrollers, reduced instruction set circuits (RISC), application specific integrated circuits (ASIC), programmable logic circuits, and any other circuit or processor capable of executing the functions described herein. The above examples are exemplary only, and thus are not intended to limit in any way the definition and/or meaning of the term processor.

When introducing elements of aspects of the disclosure or embodiments thereof, the articles “a,” “an,” “the,” and “said” are intended to mean that there are one or more of the elements. The terms “comprising,” including,” and “having” are intended to be inclusive and mean that there may be additional elements other than the listed elements.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method comprising:
establishing communication with one or more electrodes for sensing brain signals of a subject;
acquiring the brain signals via the electrodes while the subject performs at least one cognitive task, the acquired brain signals having a plurality of frequencies associated therewith;
identifying, from the acquired brain signals, an physiologic change at one or more of the plurality of frequencies; and
associating the one or more of the plurality of frequencies with the cognitive task.

2. The method of claim 1, wherein acquiring the brain signals comprises acquiring signals at a single portion of the brain.

3. The method of claim 1, wherein acquiring the brain signals comprises acquiring signals at multiple portions of the brain.

4. The method of claim 1, wherein associating the one or more of the plurality of frequencies with the cognitive task comprises detecting one of an amplitude change that is associated with the cognitive task and a phase change that is associated with the cognitive task.

5. The method of claim 1, wherein the cognitive task is one of a motor task, a speech task, an attention task, a visual task, and a memory task.

6. The method of claim 1, further comprising transmitting a signal representative of the one or more of the plurality of frequencies associated with the cognitive task to a processor.

7. The method of claim 6, further comprising decoding the signal to determine the cognitive task.

8. The method of claim 7, further comprising generating a control signal based on the cognitive task and controlling a device using the control signal.

9. An apparatus comprising:
a memory area configured to store a correlation between frequency signatures and cognitive tasks;
an interface configured to receive brain signals from a subject via one or more electrodes; and
a processor configured to:
detect, from the brain signals received by the interface, at least one of the frequency signatures; and
identify at least one of the cognitive tasks correlating to the detected frequency signature.

10. The apparatus of claim 9, wherein the interface is configured to receive the brain signals from a single portion of the brain or from multiple portions of the brain.

11. The apparatus of claim 9, wherein the processor is further configured to generate a control signal based on the identified cognitive task.

12. The apparatus of claim 11, wherein the processor is further configured to control a device using the control signal.

13. The apparatus of claim 9, wherein the processor is further configured to store the frequency signatures in the memory area and to detect changes in the frequency signatures associated with at least one cognitive task over time.

14. The apparatus of claim 9, wherein the processor is configured to detect the at least one of the frequency signatures by detecting a physiologic change within the brain signals representative of the associated cognitive task, wherein the physiologic change in the brain signals is selected from the group consisting of amplitude changes, frequency power changes, frequency phase changes, and event-related potential changes.

15. The apparatus of claim 9, wherein the brain signals are signals selected from the group consisting of electrocorticographic (ECoG) signals, electroencephalography (EEG) signals, local field potentials, single neuron signals, magnetoencephalography (MEG) signals, mu rhythm signals, beta rhythm signals, low gamma rhythm signals, and high gamma rhythm signals.

16. One or more computer-readable storage media having computer-executable components, the components comprising:
a communication component that when executed by at least one processor causes the at least one processor to receive brain signals from a subject via one or more electrodes; and
a signal analysis component that when executed by at least one processor causes the at least one processor to:
detect at least one frequency signature from the brain signals; and
identify at least one cognitive task associated with the at least one frequency signature; and.
a control component that when executed by at least one processor causes the at least one processor to perform an action related to the at least one cognitive task.

17. The computer-readable storage media of claim 16, wherein the communication component causes the at least one processor to receive the brain signals from a single portion of the brain or from multiple portions of the brain.

18. The computer-readable storage media of claim 16, wherein the signal analysis component causes the at least one processor to store the at least one frequency signature in a memory area and to detect changes in the frequency signature associated with the at least one cognitive task over time.

19. The computer-readable storage media of claim 16, wherein the signal analysis component causes the at least one processor to detect the at least one frequency signature by detecting a physiologic change within the brain signals representative of the particular action, wherein the physiologic change in the brain signals is selected from the group consisting of amplitude changes, frequency power changes, frequency phase changes, and event-related potential changes.

20. The computer-readable storage media of claim 16, wherein the brain signals are signals selected from the group consisting of electrocorticographic (ECoG) signals, electroencephalography (EEG) signals, local field potentials, single neuron signals, magnetoencephalography (MEG) signals, mu rhythm signals, beta rhythm signals, low gamma rhythm signals, and high gamma rhythm signals.
